(19) United States
(12) Patent Application Publication
(10) Pub. No.: US 2021/0267820 A1
(43) Pub. Date: Sep. 2, 2021

(54) FORMED FILMS AND ABSORBENT ARTICLES INCLUDING SAME

(71) Applicant: FITESA FILM PRODUCTS LLC, Simpsonville, SC (US)

(72) Inventors: Antonietta SPLENDIANI, Glen Allen, VA (US); Jesse B. SCHALBURG, Simpsonville, SC (US); Andrew D. MASCHINO, Paris, IL (US); Todd R. SKOCHDOPOLE, Moseley, VA (US)

(21) Appl. No.: 17/321,105

(22) Filed: May 14, 2021

Related U.S. Application Data
(63) Continuation of application No. PCT/US2019/061432, filed on Nov. 14, 2019.
(60) Provisional application No. 62/768,551, filed on Nov. 16, 2018.

(51) Int. Cl. A61F 13/511

(52) U.S. Cl. CPC A61F 13/51108; A61F 13/15658

(57) ABSTRACT

A formed film that may be used in an absorbent article. The formed film includes a plurality of wavy ridges extending generally in a machine direction. The plurality of wavy ridges form a repeating pattern of peaks and valleys. A plurality of gathering pockets are in between adjacent wavy ridges and have an apertured bottom surface offset from top surfaces of the wavy ridges in a z-direction. Each of the gathering pockets has a length in the machine direction greater than a width in a cross direction, orthogonal to the machine direction.

### CROSS-REFERENCE TO RELATED APPLICATIONS

This application is a continuation of International Patent Application No. PCT/US2019/061432, filed on Nov. 14, 2019, and relies for priority on U.S. Provisional Patent Application Ser. No. 62/768,551, filed Nov. 16, 2018, the entire contents of both of which are hereby incorporated by reference. Additionally, this application is related to U.S. patent application Ser. No. 15/584,968, filed May 2, 2017 and published as U.S. Patent Application Publication No. 2017/0312143 on Nov. 2, 2017, the entire content of which is incorporated herein by reference.

### FIELD OF THE INVENTION

The present invention is related to formed films that may be used in absorbent articles and absorbent articles that includes such formed films.

### BACKGROUND OF THE INVENTION

Formed films are used in end-products such as absorbent articles, including feminine hygiene products, adult incontinence products, and baby diapers, for example. One type of formed film that may be used in such products is a so-called topsheet, which is a top layer of the end-product that contacts the skin of the user (wearer) of the end-product. It is desirable for a topsheet that is made from a plastic film to have the visual appearance and softness of a soft cloth, instead of a stiff plastic film. However, trade-offs exist between achieving softness and a pleasant visual appearance (e.g. pattern visibility, low gloss, etc.), and the convertibility of the topsheet into the end-product, as well as the performance of the topsheet (e.g. surface distribution, wetness, masking, rewet, etc.) in the end-product when worn by the user.

The visual appearance of a topsheet, including the patterns and sizes of apertures, is generated by “thermal” forming or thermoforming, which may include vacuum-forming, hydro-forming, embossing, mechanical perforating etc., of a web of material. In vacuum-forming and hydro-forming processes, a web is deposited on a rotating forming structure, such as a screen, that includes openings corresponding to a desired pattern. In a vacuum-forming process, a relative vacuum is established across the forming structure so that the web is drawn into the openings, thereby forming a series of protuberances on the film surface. If the vacuum differential is sufficient, an opening such as a micro-aperture, may be formed in the web at the apex of each protuberance. In a hydro-forming process, similar protuberances/micro-apertures may be formed by directing a high pressure water stream at the side of the web that is opposite the forming structure. The pressure of the water stream forces the web into the opening of the forming structure. If sufficient pressure is applied, an aperture is formed in the web at the apex of each protuberance. Apertures may also be formed using mechanical methods such as needle punching, but such methods may require additional steps to provide the three dimensionality that tends to enhance the perceived softness of the final film.

A visual appearance that is appealing to users is traditionally generated by a large hydraulic radius, which should also promote absorption, and consequently a relatively high caliper or thickness, which may make the film more difficult to convert into the final end-product. Softness may be generated by softer polymers, flexible micro-structures, and cushioning structures. However, forming the webs of materials at high temperatures may make the resulting films stiffer and less soft. Conversely, softer films tend to be more difficult to convert into the final product.

In general, topsheets that are made from plastic films have better performance characteristics when used in the end-product as compared to topsheets that are made from non-woven materials. However, a topsheet made from a plastic film may have a visual appearance that is higher in gloss and therefore may be more “plastic-looking” than a non-woven topsheet, and a plastic film topsheet may feel more “sticky” or “tacky” to the wearer than a non-woven topsheet.

Typical apertured topsheets generally create a visual appearance by thermoforming holes having round, elongated, hexagonal and/or slit shapes, but challenges exist to create a unique and effective visual appearance. On the other hand, embossed topsheets may have unique visual appearances due to the laser engraving technology that is used to generate such structures, but embossed topsheets generally have very small hydraulic radii and lower performance as compared to apertured topsheets. In addition, higher basis weights are typically needed for embossed topsheets to obtain visual appearances that are more appealing to the user.

Regardless of the type of topsheet, it is desirable for the topsheet to convey a fluid insult away from the user and into an absorbent core as quickly as possible and to minimize any leakage that may occur if the fluid remains on a top surface of the topsheet.

### SUMMARY OF THE INVENTION

It is an aspect of the present invention to provide a formed film that includes a plurality of wavy ridges extending generally in a machine direction. The plurality of wavy ridges form a repeating pattern of peaks and valleys and include a first wavy ridge, a second wavy ridge, and a third wavy ridge. A peak of the first wavy ridge coincides with a valley of the second wavy ridge, and a valley of the first wavy ridge coincides with a peak of the third wavy ridge to form a continuous network of tunnels beneath the plurality of wavy ridges. The formed film also includes a plurality of first gathering pockets in between the first wavy ridge and the second wavy ridge. The first gathering pockets have an apertured bottom surface offset from top surfaces of the plurality of wavy ridges in a z-direction. The formed film also includes a plurality of second gathering pockets in between the first wavy ridge and the third wavy ridge. The second gathering pockets have an apertured bottom surface offset from the top surfaces of the plurality of wavy ridges in the z-direction. Each of the first gathering pockets and each of the second gathering pockets having a length in the machine direction greater than a width in a cross direction, orthogonal to the machine direction.

In an embodiment, the plurality of wavy ridges comprise a plurality of apertured protuberances. In an embodiment, the apertured protuberances are arranged in a 40 to 120 mesh pattern. In an embodiment, the apertured protuberances are arranged in an 80 mesh pattern. In an embodiment, the apertured protuberances are arranged in a 100 mesh pattern.

According to an aspect of the invention, there is provided an absorbent article that includes a topsheet configured to contact skin of a user of the absorbent article when the absorbent article is worn by the user. The topsheet a formed film that includes a plurality of wavy ridges extending generally in a machine direction. The plurality of wavy ridges form a repeating pattern of peaks and valleys and include a first wavy ridge, a second wavy ridge, and a third wavy ridge. A peak of the first wavy ridge coincides with a valley of the second wavy ridge, and a valley of the first wavy ridge coincides with a peak of the third wavy ridge to form a continuous network of tunnels beneath the plurality of wavy ridges. The formed film also includes a plurality of first gathering pockets in between the first wavy ridge and the second wavy ridge. The first gathering pockets have an apertured bottom surface offset from top surfaces of the plurality of wavy ridges in a z-direction. The formed film also includes a plurality of second gathering pockets in between the first wavy ridge and the third wavy ridge. The second gathering pockets have an apertured bottom surface offset from the top surfaces of the plurality of wavy ridges in the z-direction. Each of the first gathering pockets and each of the second gathering pockets having a length in the machine direction greater than a width in a cross direction, orthogonal to the machine direction. The absorbent article also includes a liquid impervious backsheet configured to be in contact with a garment worn by the user, and an absorbent core positioned in between the topsheet and the backsheet.

In an embodiment, the absorbent article also includes a sublayer positioned in between the topsheet and the absorbent core.

These and other aspects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

The components of the following figures are illustrated to emphasize the general principles of the present disclosure and are not necessarily drawn to scale. Reference characters designating corresponding components are repeated as necessary throughout the figures for the sake of consistency and clarity.

FIG. 1 schematically illustrates an absorbent article according to embodiments of the invention;

FIG. 2 illustrates a portion of a formed film according to an embodiment of the invention;

FIG. 3 schematically illustrates an apparatus for manufacturing formed films according to embodiments of the invention;

FIG. 4 schematically illustrates an apparatus for manufacturing formed films according to embodiments of the invention;

FIG. 5 illustrates a portion of a formed film according to an embodiment of the invention;

FIGS. 6A-6F illustrate a fluid insult on the absorbent article of FIG. 1 that includes the formed film of FIG. 2 over time;

FIG. 7 illustrates a comparative example of an absorbent article after the absorbent article received multiple fluid insults;

FIG. 8 illustrates a comparative example of an absorbent article after the absorbent article received multiple fluid insults;

FIG. 9 illustrates an example of an absorbent article according to an embodiment of the invention after the absorbent article received multiple fluid insults;

FIG. 10 illustrates an example of an absorbent article according to an embodiment of the invention after the absorbent article received multiple fluid insults; and

FIG. 11 illustrates an example of an absorbent article according to an embodiment of the invention after the absorbent article received multiple fluid insults.

### DETAILED DESCRIPTION OF EMBODIMENT(S) OF THE INVENTION

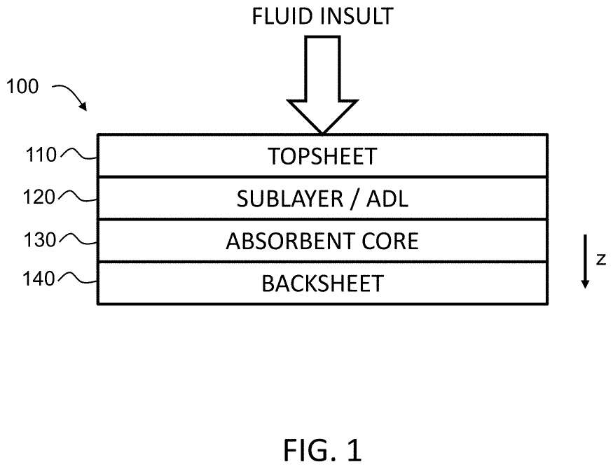

FIG. 1 is a schematic illustration of an absorbent article 100 according to embodiments of the invention. The absorbent article may be a feminine hygiene napkin, a baby diaper, or an adult incontinence product. As illustrated, the absorbent article 100 includes a topsheet 110, a sublayer 120, which may be an acquisition distribution layer (“ADL”) or transfer layer located beneath the topsheet 110, an absorbent core 130 located beneath the sublayer 120, and a backsheet 140, which may be breathable but is impervious to liquids. The topsheet 110 and/or sublayer 120 may be one of the formed films described herein in accordance with embodiments of the invention.

The topsheet 110 is user facing and in contact with the user of the absorbent article 100 when worn by the user and a fluid insult is received by the absorbent article 100 via the topsheet. Fluid that is received by the topsheet 110 passes through the topsheet 110, as described in further detail below, to the sublayer 120. The sublayer 120 may be a nonwoven material or another layer of a formed film. The sublayer 120 may be configured to distribute the fluid laterally and pass the fluid to the absorbent core 130. The backsheet 140 is in contact with an opposite side of the absorbent core 130 and is configured to keep the fluid from leaking out of the bottom side of the absorbent core 130 to the user's garment.

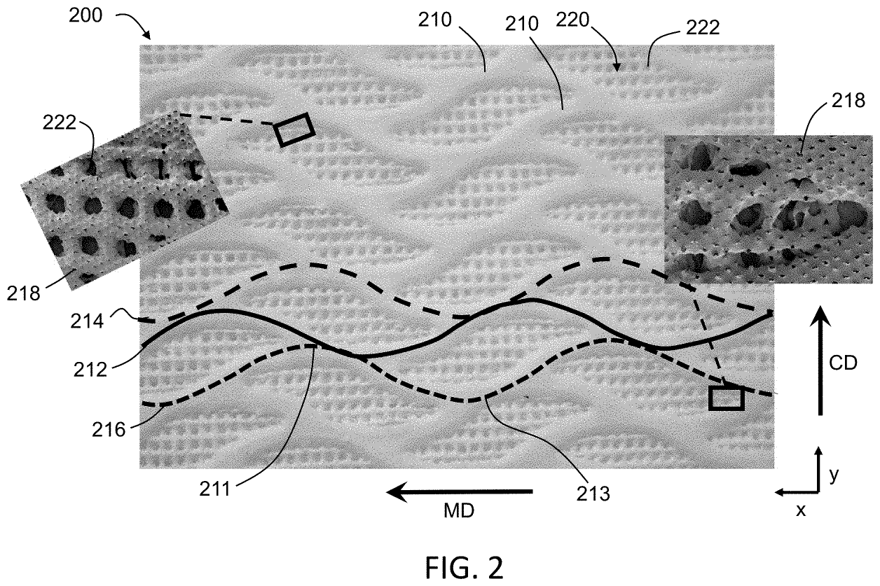

FIG. 2 illustrates a portion of a formed film 200 according to an embodiment of the present invention that may be used as the topsheet 110, or in some embodiments as the sublayer/ADL 120 of the absorbent article 100 of FIG. 1. As illustrated, the formed film 200 includes a plurality of wavy ridges 210 extending generally in a machine direction MD. The plurality of wavy ridges 210 forming a repeating pattern of peaks 211 and valleys 213. The plurality of wavy ridges includes a first wavy ridge 212, a second wavy ridge 214, and a third wavy ridge 216. Peaks of the first wavy ridge 212 are adjacent to valleys of the second wavy ridge 214, and valleys of the first wavy ridge 212 are adjacent to peaks of the third wavy ridge 216 to form a continuous network of tunnels beneath the plurality of wavy ridges 210. Each of the plurality of ridges 210 may also include a plurality of apertured protuberances 218. In the illustrated embodiment, each of the apertured protuberances 218 has a male side oriented upward towards the user and configured to contact the skin of the user. The plurality of apertured protuberances 218 may be arranged in a 40 to 120 mesh pattern. “Mesh” or “mesh pattern” as used herein is defined as the number of apertured protuberances per linear inch. In an embodiment, the apertured protuberances 218 may be arranged in an 80 mesh pattern. In an embodiment, the apertured protuberances 218 may be arranged in a 100 mesh pattern.

The formed film 200 also includes a plurality of gathering pockets 220 in between adjacent wavy ridges 210. Each gathering pocket 220 has a bottom surface that includes a plurality of apertures 222 (i.e., an apertured bottom surface) and is offset from top surfaces of the plurality of wavy ridges 210 in a z-direction. The apertures 222 may be arranged in a 15 to 45 mesh pattern, for example. In addition, the bottom surface also includes the same apertured protuberances 218 that are located on the ridges 210. In the illustrated embodiment, each aperture 222 has a generally square shape. Each of the plurality of gathering pockets 220 has a length in the machine direction MD greater than a width in a cross direction CD that is orthogonal to the machine direction MD. The ratio of the length to the width of a pocket 520 may be in the range of about 2 to about 5. The illustrated embodiment is not intended to be limiting in any way. A thermoforming process may be used to generate the plurality of apertured protuberances 218, as well as the plurality of apertures 222 in the bottom surfaces of the gathering pockets 220, and to form the plurality of gathering pockets 220 and the plurality of ridges 210, as described in further detail below.

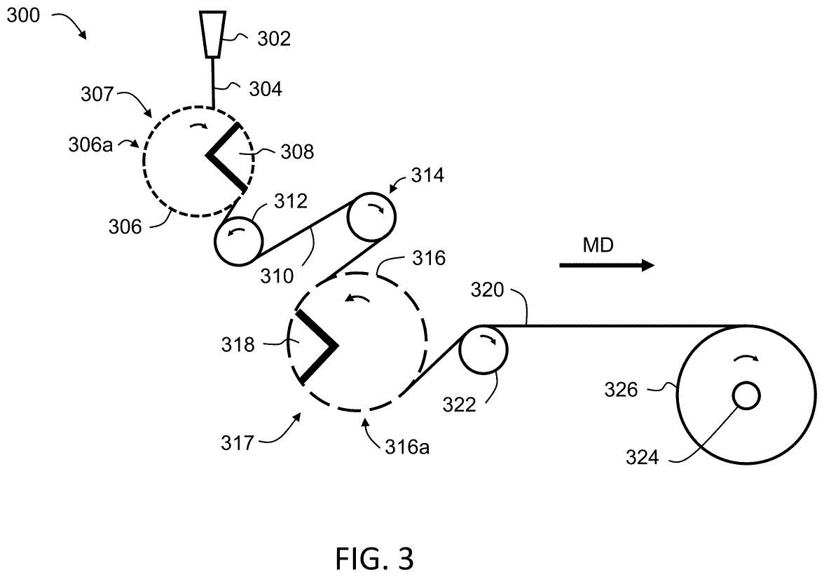

FIG. 3 schematically illustrates an embodiment of a vacuum forming apparatus 300 that may be used to manufacture formed films in accordance with embodiments of the invention. As illustrated, the apparatus 300 includes an extrusion die 302 that is located at the end of an extruder (not shown) and configured to form a polymer web 304 or extrudate. The polymer web 304 may be a single layer or a multi-layer polymer web, as would be understood by one of ordinary skill in the art. Polymers used to make the polymer web 304 may include one or more polyolefins, including but not limited to polyethylene, ultra-low density polyethylene, polypropylene, ethylene-vinyl acetates, metallocene, linear low density and linear medium density polyethylene, as well as other polymers, including but not limited to elastomeric polymers, including but not limited to polypropylene based elastomers, ethylene based elastomers, copolyester based elastomers, olefin block copolymers, styrenic block copolymers and the like, or combinations thereof. Additives, such as colorants, surfactants, etc. may be added to the polymers used to make the polymer web 304, as desired.

In the embodiment illustrated in FIG. 3, the polymer web 304 exits the extrusion die 302 and is deposited onto a first forming structure 306 that rotates around a fixed vacuum slot 308 in which a vacuum is pulled. The first forming structure 306 and the fixed vacuum slot 308 are part of a first forming station 307. The first forming structure 306 includes a plurality of openings 306a arranged in one or more patterns. When the polymer web 304 on the first forming structure 306 passes over the vacuum slot 308, the portions of the polymer web 304 that are directly over the openings 306a in the first forming structure 306 are pulled into the openings to form protrusions on the surface of the polymer web 304 facing the first forming structure 306, and if the vacuum being pulled is high enough (more negative), apertures are formed at the end of the protrusions to form an apertured web 310. The apertured web 310 is pulled off of the first forming structure 306 by a roller 312, and travels to another roller 314 before being conveyed to a second forming structure 316 that is part of a second forming station 317. The arrangement of the two rollers 312, 314 allows the apertured web 310 to be inverted so that the surface of the web that was in contact with the first forming structure 306 does not contact the second forming structure 316.

The second forming structure 316 includes a plurality of openings 316a that are arranged in one or more different patterns than the plurality of openings 306a in the first forming structure 306. As the apertured web 310 on the second forming structure 316 passes over a fixed vacuum slot 318 in which a vacuum is pulled, portions of the apertured web 310 are pulled into the plurality of openings 316a to create a formed film 320 having a three-dimensional structure in accordance with embodiments of the invention described herein. The formed film 320 may be conveyed by one or more rollers 322 to a winder 324 and wound into a roll 326 for later conversion into an absorbent article. Additional rollers may be used throughout the apparatus 300 to convey the polymer web to the winder 324. The illustrated embodiment is not intended to be limiting in any way. For example, in another embodiment, instead of extruding the polymer web 304 directly onto the first forming structure 306, a polymer web that had been previously extruded onto a chilled roll and quenched into a solid polymer web may be reheated and conveyed to the first forming station 307.

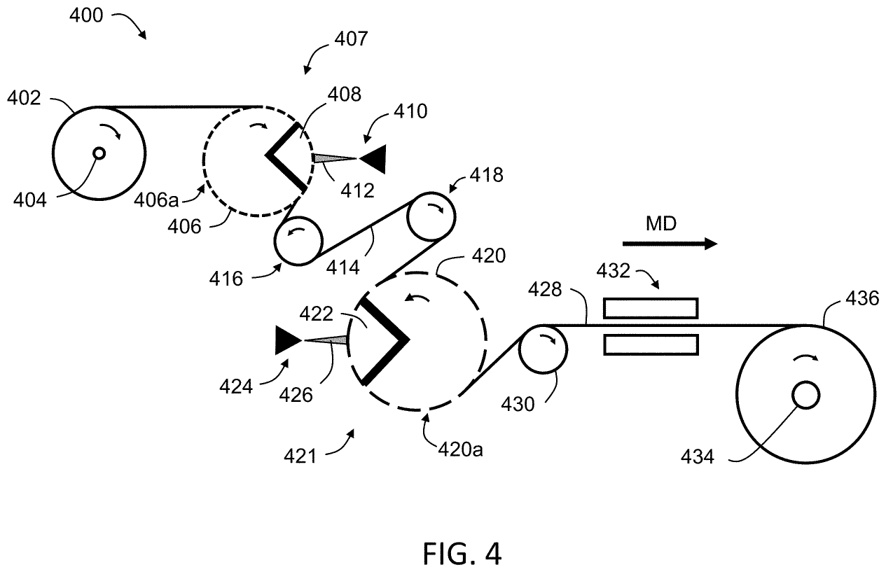

FIG. 4 illustrates an embodiment of a hydro-forming apparatus 400 that may be used to manufacture formed films in accordance with embodiments of the invention. As illustrated, a polymer web 402, which had been previously extruded and quenched may be unwound from a spindle 404 and conveyed to a first forming station 407 that includes a first forming structure 406 configured to rotate around a fixed vacuum slot 408. The first forming station 407 also includes a plurality of pressurized liquid jets 410 arranged in a long and narrow-width zone that extends into the paper containing FIG. 4, and is generally aligned with the fixed vacuum slot 408 under the forming structure 406. The liquid jets 410 are configured to provide overlapping streams of a liquid 412, such as water, at a pressure of from about 200 psi to about 800 psi onto an outer surface of the polymer web 402 while the web 402 is passing over the vacuum slot 408. In an embodiment, the liquid in the liquid jets 410 may have a pressure of from about 400 psi to about 800 psi. The liquid may be heated to help soften the polymer web 402. The streams of liquid 412 have sufficient pressure to push portions of the polymer web 402 into a plurality of openings 406a of the first forming structure 406 to form a plurality of protrusions having the same pattern as the plurality of openings 406a in the first forming structure 406. In an embodiment, the pressure is great enough to formed apertures at the end of the protrusions to form an apertured web 414. The vacuum being pulled in the vacuum slot 408 helps pull the liquid through the polymer web 402.

The apertured web 414 is pulled off of the first forming structure 406 by a roller 416, and travels to another roller 418 before being conveyed to a second forming structure 420 that is part of a second forming station 421. The arrangement of the two rollers 416, 418 allows the apertured web 414 to be inverted so that the surface of the web that was in contact with the first forming structure 406 does not contact the second forming structure 420.

The second forming structure 420 includes a plurality of openings 420a that are arranged in one or more different patterns than the plurality of openings 406a in the first forming structure 406. The second forming station 421 also includes a plurality of pressurized liquid jets 424 arranged in a long and narrow-width zone that extends into the paper containing FIG. 4, and is generally aligned with a fixed vacuum slot 422 under the forming structure 420. The liquid jets 424 are configured to provide overlapping streams of a liquid 426, such as water, at a pressure of from about 200 psi to about 800 psi onto an outer surface of the apertured web 414 while the web 414 is passing over the fixed vacuum slot 422. In an embodiment, the liquid in the liquid jets 424 may have a pressure of from about 400 psi to about 800 psi. The liquid may be heated to help soften the apertured web 414. The streams of liquid 426 have sufficient pressure to push portions of the apertured web 414 into the openings of the second forming structure 420.

As the apertured web 414 on the second forming structure 420 passes over the fixed vacuum slot 422 in which a vacuum is pulled, portions of the apertured web 414 are pulled into the plurality of openings to create a formed film 428 having a three-dimensional structure in accordance with embodiments of the invention described herein. The formed film 428 may be conveyed by one or more rollers 430 to a dryer 432 so that any residual liquid from the jets 410, 424 may be removed from the formed film 428 before being conveyed to a winder 434 and wound into a roll 436. Additional rollers may be used throughout the apparatus 400 to convey the polymer web to the winder 434. The illustrated embodiment is not intended to be limiting in any way. For example, in another embodiment, instead of unwinding the polymer web 402 from the spindle 404, the quenched polymer web may be conveyed directly from the chill roll after exiting an extrusion die to the first forming station 407. In other words, aspects of the apparatus 300 illustrated in FIG. 3 may be provided in the apparatus 400 illustrated in FIG. 4, and vice-versa.

The forming structures of the apparatus 300, 400 described above may include various meshes (openings per linear inch) and designs that may be used to create the plurality of ridges, the plurality of gathering pockets, apertures, and apertured protuberances described herein. For example, a forming structure having a mesh pattern between 40 and 120 mesh, such as 80 mesh or 100 mesh, may be used in the first forming station to form micro apertured protuberances in the polymer web. Then, a forming structure having a mesh pattern between 12 and 45 may be used as an underlying forming structure and a second forming structure having a skeleton-like structure having solid areas that correspond to the plurality of ridges and large openings in between the solid areas that correspond to the gathering pockets may be placed on top of the underlying forming structure in the second forming station. In an embodiment, the underlying forming structure and skeleton-like structure may be integrated as one forming structure for the second forming station. A combination of the openings in the underlying forming structure, and the skeleton-like structure create the porous structures at the bottoms of the gathering pockets. It should be appreciated by those of ordinary skill in the art that the embodiments of the formed film described herein will have associated forming structure designs that may be used in the apparatus 300, 400 described above.

The forming structures may have uniform patterns or may have different zones containing different patterns or no patterns. For example, different zones may have different mesh patterns that will be transferred to the polymer web.

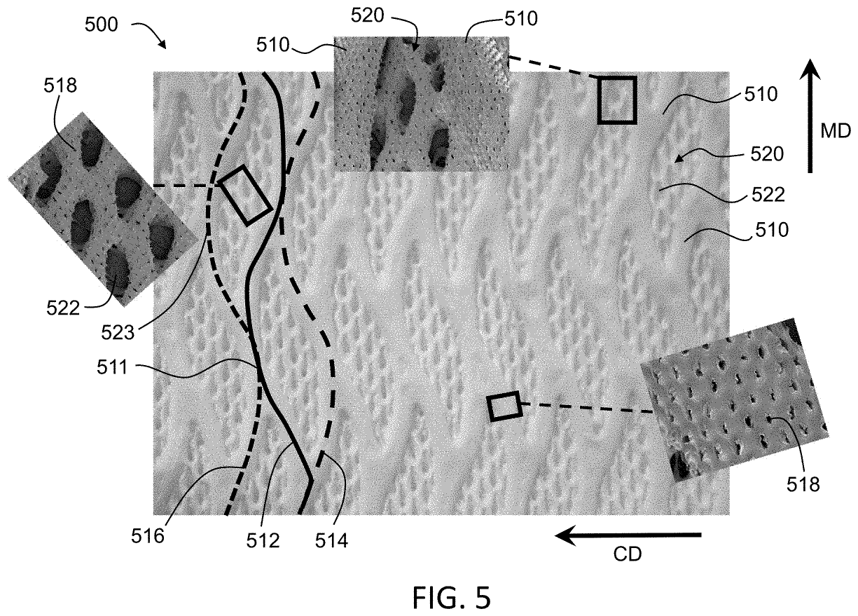

FIG. 5 illustrates a portion of a formed film 500 according to an embodiment of the present invention that may be used as the topsheet 110, or in some embodiments as the sublayer/ADL 120 of the absorbent article 100 of FIG. 1. As illustrated, the formed film 500 includes a plurality of wavy ridges 510 extending generally in a machine direction MD. The plurality of wavy ridges 510 forming a repeating pattern of peaks and valleys. The plurality of wavy ridges includes a first wavy ridge 512, a second wavy ridge 514, and a third wavy ridge 516. Peaks of the first wavy ridge 512 are adjacent to valleys of the second wavy ridge 514, and valleys of the first wavy ridge 512 are adjacent to peaks of the third wavy ridge 516 to form a continuous network of tunnels beneath the plurality of wavy ridges 510. Each of the plurality of ridges 510 may also include a plurality of apertured protuberances 518. In the illustrated embodiment, each of the apertured protuberances has a male side oriented upward towards the user and configured to contact the skin of the user. The plurality of apertured protuberances 518 may be arranged in a 40 to 120 mesh pattern. In an embodiment, the apertured protuberances 518 may be arranged in an 80 mesh pattern. In an embodiment, the apertured protuberances 518 may be arranged in a 100 mesh pattern.

The formed film 500 also includes a plurality of gathering pockets 520 in between adjacent wavy ridges 510. Each gathering pocket 520 has a bottom surface that includes a plurality of apertures 522 (i.e., an apertured bottom surface) and is offset from top surfaces of the plurality of wavy ridges 510 in a z-direction. The apertures 522 may be arranged in a 15 to 45 mesh pattern, for example. In addition, the bottom surface also includes the same apertured protuberances 518 that are located on the ridges 510. In the illustrated embodiment, each aperture 522 is generally shaped as a teardrop. Each of the plurality of gathering pockets 520 has a length in the machine direction MD greater than a width in a cross direction CD that is orthogonal to the machine direction MD. The ratio of the length to the width of a pocket 520 may be in the range of about 2 to about 5. The illustrated embodiment is not intended to be limiting in any way. A thermoforming process may be used to generate the plurality of apertured protuberances 518, as well as the plurality of apertures 522 in the bottom surfaces of the gathering pockets 520, and to form the plurality of gathering pockets 520 and the plurality of ridges 510, as described above.

### Comparative Example A

Feminine hygiene napkins sold under the brand name Equate™ were used as a control for strikethrough and rewet testing, described below. The topsheet of each of the Equate™ feminine hygiene napkins is a laminate structure that includes a nonwoven layer and a formed film layer. The formed film layer is made from polyethylene and has a fine mesh of three-dimensional micro-apertures that extend away from the non-woven layer. For Comparative Example A, the as-manufactured napkin was used, with no alterations made to the napkin.

### Comparative Example B

For a second control, the topsheet of the Equate™ feminine hygiene napkin was removed from the so-called chassis (remainder) of the napkin, placed back on top of the chassis, and re-attached to re-form the napkin.

### Example 1

The formed film 200 illustrated in FIG. 2 was cut in the shape of the topsheet of the Equate™ feminine hygiene napkin of Comparative Example B and attached to the chassis of the Equate™ feminine hygiene napkin after the topsheet of the Equate™ feminine hygiene napkin was removed.

### Example 2

The formed film 500 illustrated in FIG. 5 was cut in the shape of the topsheet of the Equate™ feminine hygiene napkin of Comparative Example B and attached to the chassis of the Equate™ feminine hygiene napkin after the topsheet of the Equate™ feminine hygiene napkin was removed.

### Example 3

A formed film having the same design as the formed film 500 illustrated in FIG. 5, but including less surfactant in the polymer blend, was cut in the shape of the topsheet of the Equate™ feminine hygiene napkin of Comparative Example B and attached to the chassis of the Equate™ feminine hygiene napkin after the topsheet of the Equate™ feminine hygiene napkin was removed.

### Testing

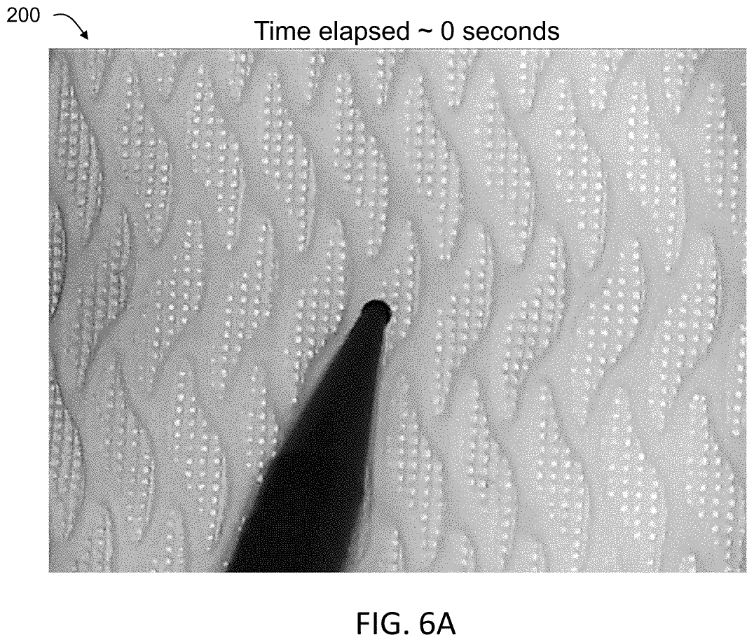

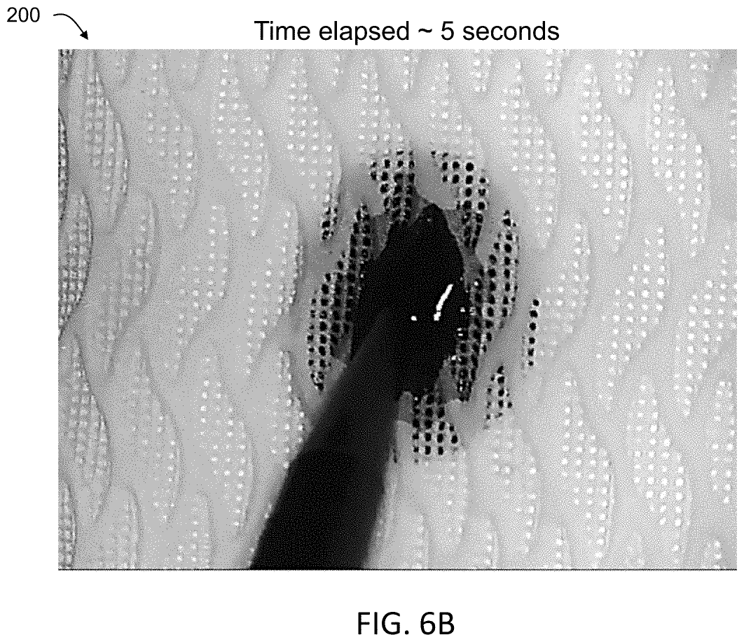

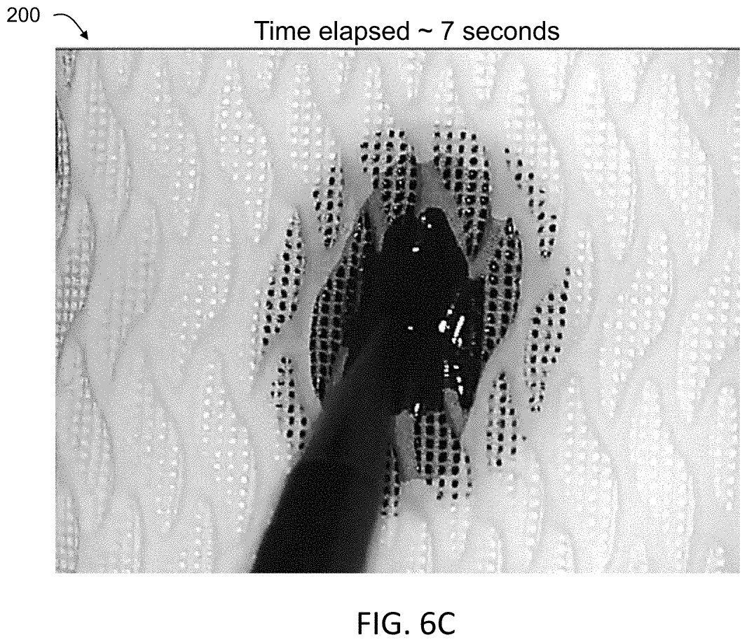

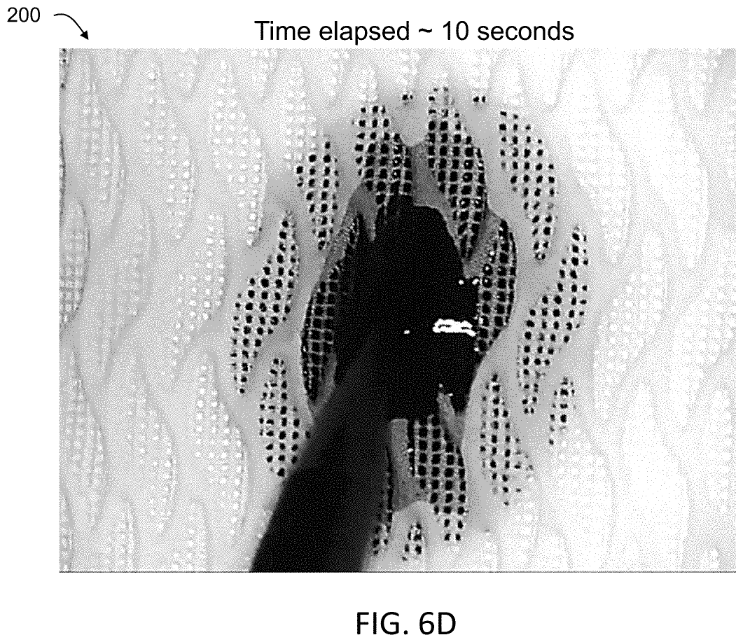

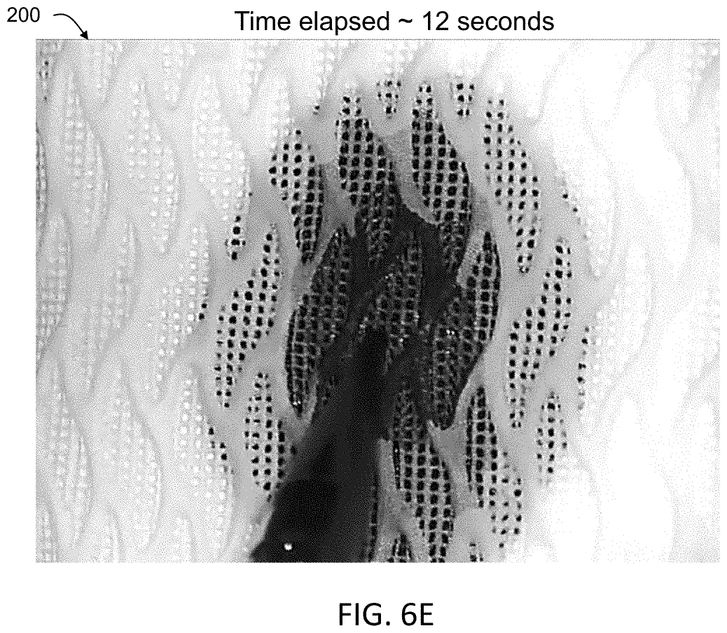

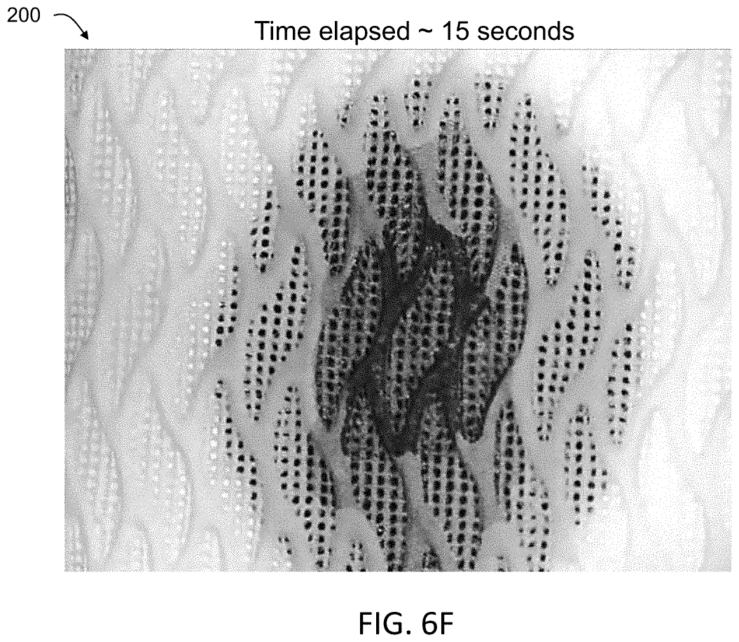

FIGS. 6A-6F illustrate an insult of synthetic blood having a viscosity of 11.1 centipoise (cP) being applied to a specimen in accordance with Example 1, and the insult passing through the topsheet and into the absorbent core of the napkin. Specifically, FIG. 6A illustrates a moment in time that the insult began to exit a delivery system (i.e., elapsed time ˜0 seconds), FIG. 6B illustrates approximately 5 seconds later (i.e., elapsed time ˜5 seconds), FIG. 6C illustrates approximately 2 seconds later (i.e., elapsed time ˜7 seconds), FIG. 6D illustrates approximately 3 seconds later (i.e., elapsed time ˜10 seconds), FIG. 6E illustrates approximately 2 seconds later (i.e., elapsed time ˜12 seconds), and FIG. 6F illustrates approximately 3 seconds later (i.e., elapsed time ˜15 seconds). As illustrated, the insult passes through the formed film topsheet and is absorbed by the absorbent core. The gathering pockets assist to pool the fluid away from the top surface of the formed film, and the ridges assist to move the fluid in the machine direction (MD) more quickly than the cross direction (CD). After the fluid has passed through the bottoms of the pockets, the tunnels beneath the ridges assist to rapidly evacuate the fluid from the insult point and drive the fluid in the machine direction (MD) of the formed film as the fluid is absorbed by the absorbent core.

Examples 1, 2 and 3, and Comparative Examples A and B, described above, were tested for performance characteristics, including multiple strikethrough times and rewet. For each example, three specimens were subjected to three insults of 4 milliliters (ml) of a synthetic blood having a viscosity of 11.2 centipoise (cP). Specifically, each specimen was placed on a flat table top, a strikethrough plate made from clear plastic with a hole in the center was place on top of the specimen so that the hole in the plate was centered on the specimen, and a pump was started to deliver the 4 ml insult in 6 seconds. A stopwatch was started at the same time the pump was started and stopped when the insult liquid in the plate hole was observed to be absorbed by the specimen.

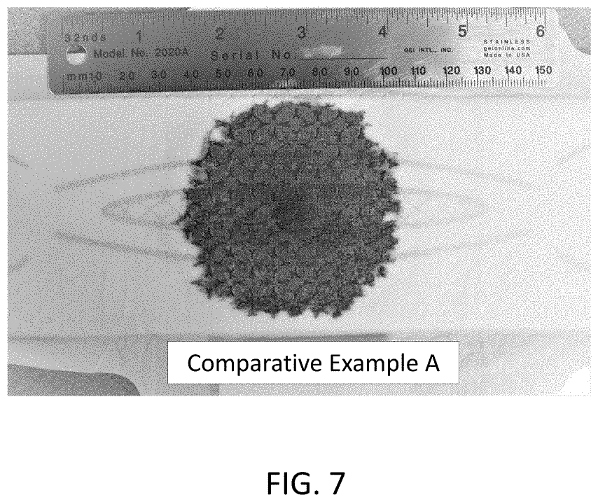

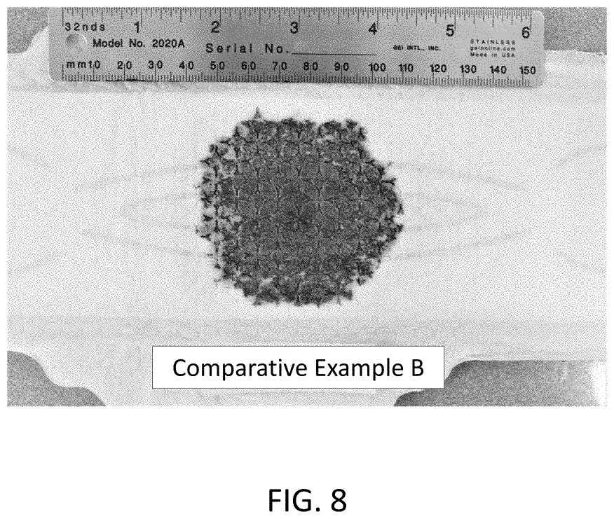

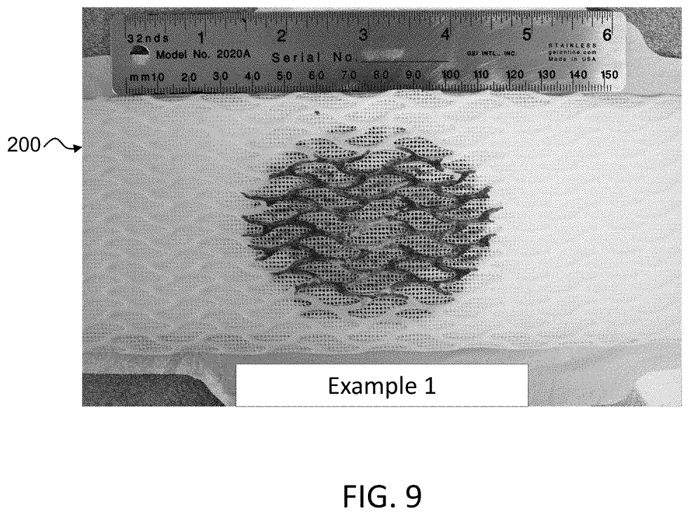

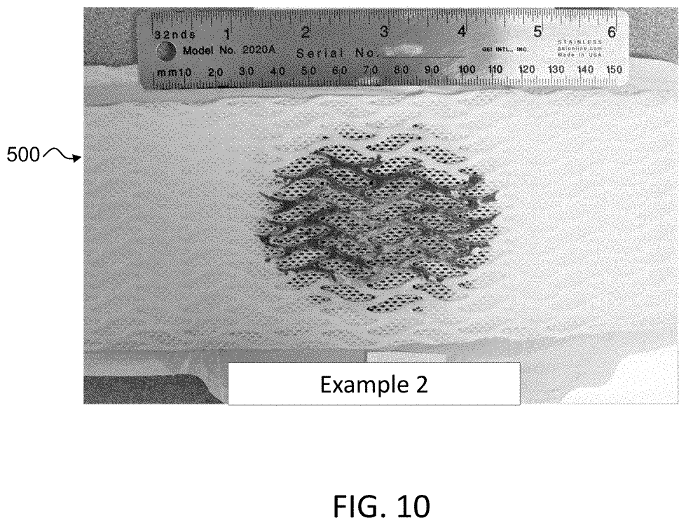

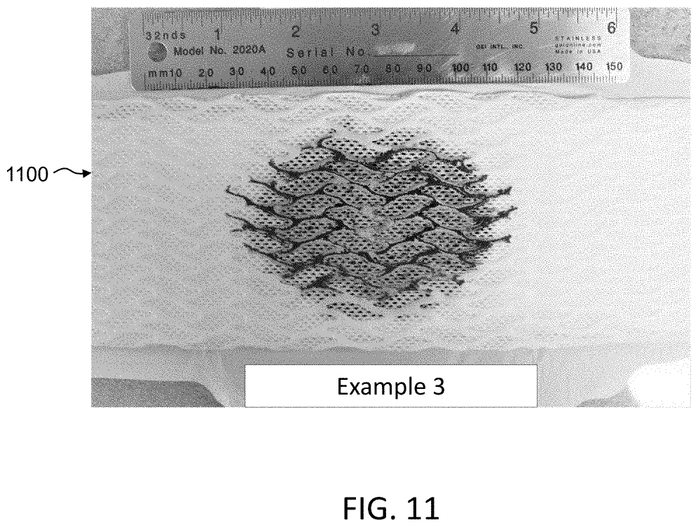

The recorded time was the first insult strikethrough time. With the strikethrough plate remaining on the specific, after a 5 minute wait, the pump and stopwatch were started and the stopwatch was stopped when the insult liquid in the plate hole was observed to be absorbed by the specimen. The recorded time was the second insult strikethrough time. With the strikethrough plate remaining on the specific, after a 5 minute wait, the pump and stopwatch were started and the stopwatch was stopped when the insult liquid in the plate hole was observed to be absorbed by the specimen. The recorded time was the third insult strikethrough time. After a 5 minute wait, the strikethrough plate was removed from the specimen and a picture of the specimen was taken. FIGS. 7-11 illustrate various specimens after the strikethrough plate was removed. Specifically, FIG. 7 illustrates one specimen of Comparative Example A, FIG. 8 illustrates one specimen of Comparative Example B, FIG. 9 illustrates one specimen of Example 1, FIG. 10 illustrates one specimen of Example 2, and FIG. 11 illustrates one specimen of Example 3.

After a picture was taken, 5 sheets of pre-weighed pickup paper were placed over the insult area and a 4.8 lb. weight, which simulated an applied pressure of 0.6 psi, was placed on top of the sheets of paper for 2 minutes. After two minutes, the weight was removed and the 5 papers were weighed. The difference between the weight of the “wet” paper and the initial “dry” paper is the final rewet value in grams. The results of the strikethrough and rewet tests (averages of three specimens each) are summarized in Table I.

TABLE I
STRIKETHROUGH & REWET TEST RESULTS

| | Strikethrough (sec) | | | |
|---|---|---|---|---|
| | First | Second | Third | Rewet |
| Test Sample | Insult | Insult | Insult | (g) |
| Comparative | 52.83 | 201.09 | 286.45 | 3.21 |
| Example A | | | | |
| Comparative | 46.73 | 152.68 | 259.14 | 3.26 |
| Example B | | | | |
| Example 1 | 18.62 | 44.74 | 52.68 | 2.90 |
| Example 2 | 16.95 | 40.61 | 47.18 | 3.09 |
| Example 3 | 15.57 | 36.29 | 42.91 | 3.21 |

The strikethrough test results indicate that when the formed films of the present invention (i.e., Examples 1-3) are used as a topsheet for a feminine hygiene article, the strikethrough times are significantly lower than the strikethrough times exhibited by the comparative examples, and the differences are more pronounced after multiple insults. The rewet test results indicate that the formed films of the present invention provide similar rewet values as the comparative examples.

The pictures that were taken at the end of the strikethrough test, and before the rewet measurement, were used to measure the size of the stain on the top surface of the topsheet (not the size of the stain in the absorbent core). Specifically, the size of the stain in the machine direction (MD) and the cross direction (CD) were measured in millimeters (mm) using a ruler, and a ratio between the two measurements was calculated. FIGS. 7-11 illustrate the ruler extending in the machine direction (MD). The results are summarized in Table II.

TABLE II
STAIN MEASUREMENT RESULTS

| | MD Length | CD Length | MD/CD |
|---|---|---|---|
| Test Sample | (mm) | (mm) | Ratio |
| Comparative | 67 | 65 | 1.02 |
| Example A | | | |
| Comparative | 68 | 63 | 1.07 |
| Example B | | | |
| Example 1 | 80 | 49 | 1.65 |
| Example 2 | 78 | 46 | 1.70 |
| Example 3 | 88 | 50 | 1.75 |

A ratio of 1 indicates a stain in the general shape of a circle and a ratio>1 indicates a stain in the general shape of an ellipse, with the long axis in the machine direction. As illustrated in FIGS. 7 and 8 and listed in Table II, the Comparative Examples had stains on their top surfaces in the general shape of a circle, while all three Examples had stains on their top surfaces in the general shape of an ellipse, which is more desirable. Without being bound by theory, it is hypothesized that a more elongated stain on the top surface in the MD (i.e., higher MD/CD ratio) indicates that the topsheet will be less likely to leak in the CD.

Formed films in accordance with embodiments of the invention may be manufactured using thermoforming processes, including but not limited to vacuum forming, hydro-forming, mechanical forming, embossing, and/or combinations thereof. Preferably, the formed films in accordance with embodiments of the invention are manufactured using a hydro-forming process and apparatus 400 illustrated in FIG. 4, for example.

Although the embodiments of the invention have been described above with respect to the formed film being used as a topsheet for an absorbent article, it is contemplated that the formed film may also be used as a sublayer or acquisition distribution layer. The illustrated embodiments are not intended to be limiting in any way.

The embodiments described herein represent a number of possible implementations and examples and are not intended to necessarily limit the present disclosure to any specific embodiments. Instead, various modifications can be made to these embodiments as would be understood by one of ordinary skill in the art. Any such modifications are intended to be included within the spirit and scope of the present disclosure and protected by the following claims.

What is claimed is:

1. A formed film comprising:
a plurality of wavy ridges extending generally in a machine direction, the plurality of wavy ridges forming a repeating pattern of peaks and valleys and comprising a first wavy ridge, a second wavy ridge, and a third wavy ridge, wherein a peak of the first wavy ridge coincides with a valley of the second wavy ridge, and a valley of the first wavy ridge coincides with a peak of the third wavy ridge to form a continuous network of tunnels beneath the plurality of wavy ridges; and
a plurality of first gathering pockets in between the first wavy ridge and the second wavy ridge, the first gathering pockets having an apertured bottom surface offset from top surfaces of the plurality of wavy ridges in a z-direction;
a plurality of second gathering pockets in between the first wavy ridge and the third wavy ridge, the second gathering pockets having an apertured bottom surface offset from the top surfaces of the plurality of wavy ridges in the z-direction, each of the first gathering pockets and each of the second gathering pockets having a length in the machine direction greater than a width in a cross direction, orthogonal to the machine direction.

2. The formed film according to claim 1, wherein the plurality of wavy ridges comprise a plurality of apertured protuberances.

3. The formed film according to claim 2, wherein the apertured protuberances are arranged in a 40 to 120 mesh pattern.

4. The formed film according to claim 3, wherein the apertured protuberances are arranged in an 80 mesh pattern.

5. The formed film according to claim 3, wherein the apertured protuberances are arranged in a 100 mesh pattern.

6. An absorbent article comprising:
a topsheet configured to contact skin of a user of the absorbent article when the absorbent article is worn by the user, the topsheet comprising the formed film according to claim 1;
a liquid impervious backsheet configured to be in contact with a garment worn by the user; and
an absorbent core positioned in between the topsheet and the backsheet.

7. The absorbent article according to claim 6, further comprising a sublayer positioned in between the topsheet and the absorbent core.
